(19) United States
(12) Patent Application Publication
(10) Pub. No.: US 2022/0290141 A1
(43) Pub. Date: Sep. 15, 2022

(54) ANTISENSE OLIGONUCLEOTIDE THERAPY FOR CANCER

(71) Applicant: MURDOCH UNIVERSITY, Murdoch, Western Australia (AU)

(72) Inventors: Rakesh Naduvile Veedu, Australia (AU); Suxiang Chen, Guanzhou City, Guangdong (CN)

(21) Appl. No.: 17/638,041

(22) PCT Filed: Sep. 2, 2020
(86) PCT No.: PCT/AU2020/050919

§ 371 Date: Feb. 24, 2022

(30) Foreign Application Priority Data
Sep. 3, 2019 (AU) 2019903247

(51) Int. Cl. C12N 15/113; A61K 31/7125; A61P 35/00

(52) U.S. Cl. CPC C12N 15/113; A61K 31/7125; A61P 35/00; C12N 2310/11; C12N 2310/31; C12N 2310/33; C12N 2320/33

(57) ABSTRACT

An isolated or purified antisense oligomer for modifying pre-mRNA splicing in the LIN28B gene transcript or part thereof which has a modified backbone structure and sequences with at least 95% sequence identity to the isolated or purified antisense oligomer which have a modified backbone structure for modifying pre-mRNA splicing, and/or induction of RNase H, and/or translational blockage in the LIN28B gene transcript or part thereof.

### TECHNICAL FIELD

The present invention relates to the use of antisense oligomers targeting LIN28B to treat cancers, particularly solid tumour cancers.

### BACKGROUND ART

LIN28B is an RNA binding protein highly expressed during embryogenesis, and generally the protein is not expressed after cell differentiation and body development.

Recently, it has been reported that LIN28B is over-expressed in various solid cancers (e.g. liver cancer, brain cancer). LIN28B over-expression represses let-7 microRNA biogenesis, and Let-7 negatively regulates the translation of oncogenes like c-Myc, Kras, and Hmga2. Over-expression of LIN28B is linked to PD-L1 over-expression. Reports also show that LIN28B overexpression is sufficient to initiate hepatoblastoma and hepatocellular carcinoma (HCC) in murine models, and that liver-specific deletion of Lin28a/b reduced tumour burden, extended latency, and prolonged survival. Thus, LIN28B is a potential therapeutic target for developing cancer therapeutics, particularly against solid tumours including liver cancer and brain cancer.

Liver cancer is the 5th most prominent cancer worldwide, and its numbers are increasing due to factors like obesity, alcohol consumption and drug use. Primary liver cancer, unlike most other cancers, usually develops from chronic liver damage observed in conditions including alcoholic liver disease (ALD), hepatitis B virus (HBV) and non-alcoholic fatty liver disease (NAFLD). Hepatocellular carcinoma (HCC) is the most common liver cancer, accounting for 90% of primary liver cancers. Due to disease severity and the lack of effective treatments, worldwide liver cancer is increasing and is the second highest cause of cancer-related deaths. The financial cost of liver disease in Australia is enormous and in 2012 was estimated at $5.4 billion and the total economic cost including the burden of disease was estimated at $50.7 billion. Indeed, the total cost of liver diseases was approximately 40% higher than Type 2 diabetes and chronic kidney diseases combined. The dramatic rising incidence of liver disease and cancer provides compelling evidence that there is a significant need to develop new therapies for treating liver diseases and liver cancer; or at least the provision of new therapies to compliment the previously known cancer therapies.

The current treatment approaches for liver cancer and other solid cancers include chemotherapeutics (small-molecule chemicals, antibodies etc.), surgery, and radiation therapy.

The present invention seeks to provide an improved or alternative method for the treatment of cancers associated with expression of LIN28B.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

Broadly, according to one aspect of the invention, there is provided an isolated or purified antisense oligonucleotide (ASO) for interfering with the normal function of cellular RNA (including pre-mRNA and/or mRNA) in the LIN28B gene transcript or part thereof. The functions of RNA to be interfered with include all vital functions such as, for example, splicing of the pre-mRNA to yield one or more mRNA transcripts, translocation of the RNA to the site of protein translation, translation of protein from the RNA, and catalytic activity which may be engaged in or facilitated by the RNA. The overall effect of such interference with target RNA function is inhibition or decrease of the expression of LIN28B. Preferably, there is provided an isolated or purified antisense oligomer for inducing non-productive splicing in the LIN28B gene transcript or part thereof.

For example, in one aspect of the invention, there is provided an antisense oligomer of 10 to 50 nucleotides comprising a targeting sequence complementary to a region near or within an intron of the LIN28B gene transcript or part thereof. In another aspect of the invention, there is provided an antisense oligomer of 10 to 50 nucleotides comprising a targeting sequence complementary to a region near or within an exon of the LIN28B gene transcript or part thereof.

Preferably, the antisense oligomer is a full 2′-O-Methyl phosphorothioate (2′-OMe-PS), full 2′-O-Methoxyethyl phosphorothioate (2′-O-MOE-PS), 7-11-7 2′-O-Methoxyethyl phosphorothioate/DNA phosphorothioate (2′-O-MOE-PS/DNA-PS) gapmer or phosphorodiamidate morpholino oligomer (PMO). A 7-11-7 2′-O-Methoxyethyl phosphorothioate/DNA phosphorothioate (2′-O-MOE-PS/DNA-PS) gapmer is a 11 mer DNA-PS core region flanked by 7 mer 2′-MOE-PS regions at both 5′ end and 3′ end of the core region.

Preferably, the antisense oligomer is selected from the group comprising the sequences set forth in Table 1. Preferably, the antisense oligomer is selected from the list comprising: SEQ ID NOs: 1-43, more preferably SEQ ID NOs: 2, 4, 5 and 6. Most preferably, the antisense oligomer is SEQ ID NO: 2.

The antisense oligomer preferably operates to induce skipping of one or more of the exons of the LIN28B gene transcript or part thereof. For example, the antisense oligomer may induce skipping of exon-2. The antisense oligomer may operate to induce RNase H mediated cleavage of LIN28B mRNA using gapmer antisense designs. For example, the antisense oligomer may be a 7-11-7 2′-O-MOE PS/DNA PS gapmer antisense molecule targeting LIN28B mRNA to induce RNase H-mediated mRNA degradation.

The antisense oligomer of the invention may be selected to be an antisense oligomer capable of binding to a selected LIN28B target site, wherein the target site is an mRNA splicing site selected from a splice donor site, splice acceptor sites, or exonic splicing elements. The target site may also include some flanking intronic sequences when the donor or acceptor splice sites are targeted.

More specifically, the antisense oligomer may be selected from the group comprising of any one or more of SEQ ID NOs: 1-43, more preferably SEQ ID NOs: 2, 4, 5 and 6. Most preferably, the antisense oligomer is SEQ ID NO: 2 and/or the sequences set forth in Table 1, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a LIN28B gene transcript. In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 85% sequence complementarity, e.g., 85%, 86%, 87%, 88%, 89%, 90%, 91%, 92%, 93%, 94%, 95%, 96%, 97%, 98%, 99% or 100% sequence complementarity, between the oligonucleotide and the target sequence.

The invention extends also to a combination of two or more antisense oligomers capable of binding to a selected target to induce exon exclusion in a LIN28B gene transcript, including a construct comprising two or more such antisense oligomers. The construct may be used for an antisense oligomer-based therapy.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

There is also provided a method for manipulating splicing in a LIN28B gene transcript, the method including the step of:

a) providing one or more of the antisense oligomers as described herein and allowing the oligomer(s) to bind to a target nucleic acid site.

There is also provided a pharmaceutical or therapeutic composition to treat or ameliorate the effects of a cancer related to LIN28B expression in a subject, the composition comprising:

a) one or more antisense oligomers as described herein; and

b) one or more pharmaceutically acceptable carriers and/or diluents.

The composition may comprise about 1 nM to 1000 nM of each of the desired antisense oligomer(s) of the invention. Preferably, the composition may comprise about 10 nM to 500 nM, most preferably between 1 nM and 10 nM of each of the antisense oligomer(s) of the invention.

There is also provided a method to treat or ameliorate the effects of a cancer associated with LIN28B expression in a subject, comprising the step of:

a) administering to the subject an effective amount of one or more antisense oligomers or pharmaceutical composition comprising one or more antisense oligomers as described herein.

There is also provided the use of purified and isolated antisense oligomers as described herein, for the manufacture of a medicament to treat or ameliorate the effects of a cancer associated with LIN28B expression.

There is also provided a kit to treat or ameliorate the effects of a cancer associated with LIN28B expression in a subject, which kit comprises at least an antisense oligomer as described herein and combinations or cocktails thereof, packaged in a suitable container, together with instructions for its use.

Preferably the cancer associated with LIN28B expression in a subject is a solid tumour cancer. More preferably, the cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukaemia, atypical teratoid tumour, oesophageal cancer, medulloblastoma, multiple myeloma, neuroblastoma, oral squamous cell carcinoma, Wilms tumor, and prostate cancer.

The subject with the cancer associated with LIN28B expression may be a mammal, including a human.

Further aspects of the invention will now be described with reference to the accompanying non-limiting examples and drawings.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is an exon map of LIN28B.

FIG. 2(A) shows the deletion of exon-2 induces 5 premature termination codons in exon-3 and exon-4 respectively, and 2(B) shows partial deletion of exon-2 induces 1, 5, and 5 premature termination codons in dual exon-2&3, exon-3, and exon-4 respectively.

FIG. 3 is an image of the expression of LIN28B in cancer cells and normal cells. Annealing temperatures of RT-PCR reactions included 57.8° C., 60° C., and 62° C.

FIG. 4 is an image of the inhibition of LIN28B RNA. 4(A) in liver cancer and 4(B) in glioblastoma cells in vitro using antisense oligonucleotides. Transfection reagent used for the ASO transfection was RNAiMAX. Transfection concentrations of ASOs were all at 400 nM.

FIG. 5(A) is a Sanger sequencing analysis of the conformation of LIN28B exon-2 skipping using ASO-2, LIN28B 1E2A (+10+34), 5(B) is the conformation of LIN28B partial exon-2 skipping using ASO-6, LIN28B 1E2A (+142+166).

FIG. 6 is an image of the dose-dependent inhibition of LIN28B RNA by exon-2 skipping using 2′-OMe-PS chemistry form of ASO-2, LIN28B 1E2A (+10+34) in 6(A) HepG2 liver cancer cells, 6(B) U87 glioblastoma cells in vitro.

FIG. 7 is an image of the dose-dependent inhibition of LIN28B RNA by exon-2 skipping and/or partial exon-2 skipping and/or full length transcript reduction by RNase H mediated mRNA degradation, by three different chemistry designs (fully 2′-OMe-PS, fully 2′-MOE-PS, and 7-11-7 MOE PS/DNA PS gapmer) of ASO-2, ASO-4, ASO-5, and ASO-6 in liver cancer HepG2 cells.

FIG. 8 is an image of the inhibition of LIN28B protein by fully 2-MOE-PS form of ASO-2, ASO-4, ASO-5, and ASO-6 in liver cancer HepG2 cells. Final concentrations of ASOs for transfection was 400 nM. Cell density was 250 000 cells per well in a 6-well plate. Transfection reagent used was RNAiMAX. 8(A): Western blot gel image, 8(B): densitometry analysis based on the gel image.

FIG. 9 is an image of the inhibition of LIN28B RNA by PMO form of ASO-2 in liver cancer HepG2 cells and normal human liver IHH cells. Compared to the HepG2 cells, IHH cells only show very weak expression of LIN28B in untreated groups. Concentrations of PMO form of ASO-2 included 30 μM and 15 μM (concentrations in nucleofection kit cuvettes where nucleofection process was occurred, not the final concentration in wells of 6-well plate). 9(A): cells were harvested 24 hours after nucleofection; 9(B): cells were harvested 5 days after nucleofection.

FIG. 10 is a plot showing the evaluation of cell viability using LIN28B exon-2 targeting ASOs of three different chemistry designs (fully 2′-OMe-PS, fully 2′-O-MOE-PS, and 7-11-7 MOE-PS/DNA-PS gapmer). 10(A): transfection reagent used for ASO transfection was lipofectin, 10(A1): liver cancer HepG2 cells, 10(A2): normal human liver IHH cells; 10(B): transfection reagent used for ASO transfection was lipofectamine 3000 (L3K), 10(B1): liver cancer HepG2 cells, 10(B2): normal human liver IHH cells.

FIG. 11 is a plot showing the evaluation of cell viability using LIN28B exon-2 targeting ASOs of three different chemistry designs (fully 2′-OMe-PS, fully 2′-O-MOE-PS, and 7-11-7 MOE-PS/DNA-PS gapmer) in liver cancer HepG2 cells, transfection reagent used for ASO transfection was RNAiMAX. 11(A): HepG2 cell density was 25 000 cells per well in 24-well plate, 11(B): HepG2 cell density was 50 000 cells per well in 24-well plate.

FIG. 12 is an image of initial screening of ASOs targeting exon-1, exon-3, and exon-4 of LIN28B in Huh-7 cells. The Huh-7 cell density was 25 000 cells per well in 24-well plate.

### DESCRIPTION OF INVENTION

### Detailed Description of the Invention

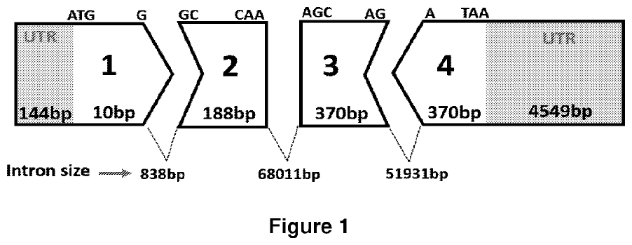

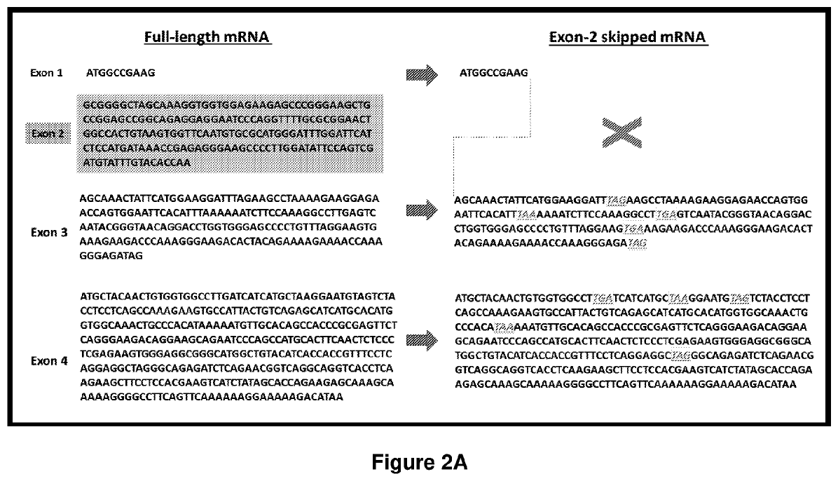

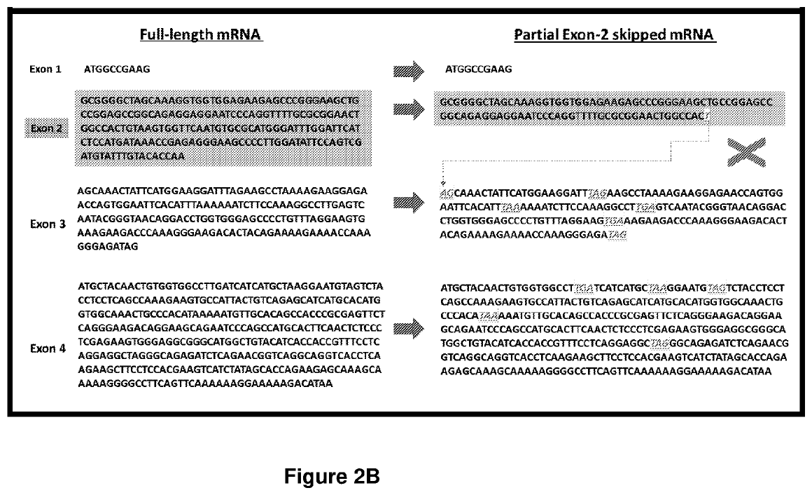

LIN28B has 4 exons (FIG. 1). The present invention has discovered that targeting exon-2 using splice modulating antisense oligonucleotides (ASOs) will induce exon-2 deletion resulting in 5 premature termination codons in exon-3 and exon-4 respectively, and/or partial deletion of exon-2 resulting in 1, 5, and 5 premature termination codons in dual exon-2 & -3, exon-3, and exon-4 respectively (FIG. 2). In addition, translational blocking and/or induction of RNase H-based degradation of LIN28B RNA using ASOs can efficiently inhibit the expression of LIN28B.

The advantage of targeting the LIN28 gene is that it is directly linked to various cancer pathological hallmark pathways. LIN28 regulates important biological processes including stem cell differentiation. However, LIN28 expression is largely restricted to embryonic development and generally not expressed after cell differentiation and body development. In tumour cells, LIN28 is frequently dysregulated and overexpressed. LIN28 interferes with conversion of pre-let-7 miRNA transcripts to mature let-7 miRNAs, thereby suppressing let-7 miRNA-mediated downstream tumour inhibition effects, as members of the let-7 miRNA family play important roles as tumour suppressors. Let-7 miRNAs bind to many key oncogenes including RAS and MYC and inhibits their expression. In addition, let-7 miRNAs also suppress tumour cell proliferation by inhibiting cell-cycle regulating genes including CDK6, E2F2, and CCND2.

The present invention therefore provides antisense oligonucleotides to induce non-productive splicing or functionally impaired protein of LIN28B to reduce or eliminate expression of LIN28B and therefore unblock the maturation of let-7 miRNAs or recover the function of let-7 miRNA-induced cancer cell inhibition effect, thus inhibiting the progress of solid cancers or tumours.

In contrast to other antisense oligomer based therapies, the present invention induces inhibition or reduction of LIN28B expression via induction of pre-mature termination codons resulting from pre-mRNA splicing, and/or degradation of mRNA via recruitment of RNase H, wherein the RNase H preferentially binds to and degrade mRNA bound in duplex to DNA, and/or translational blockage of RNA.

Preferably, the antisense oligomers are used to modify pre-mRNA splicing in a LIN28B gene transcript or part thereof and induce exon “skipping”. The strategy preferably reduces total protein expression or generates proteins which lack functional domains, leading to reduced protein function. According to a first aspect of the invention, there is provided antisense oligomers capable of binding to a selected target on a LIN28B gene transcript to modify pre-mRNA splicing in a LIN28B gene transcript or part thereof. Broadly, there is provided an isolated or purified antisense oligomer for interfering with the normal function of cellular RNA (including pre-mRNA and/or mRNA) in a LIN28B gene transcript or part thereof.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An antisense oligomer can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing. The target sequence may be within an exon or within an intron or spanning an intron/exon junction.

In certain embodiments, the antisense oligomer has sufficient sequence complementarity to a target RNA (i.e., the RNA for which splice site selection is modulated) to block a region of a target RNA (e.g., pre-mRNA) in an effective manner. In exemplary embodiments, such blocking of LIN28B pre-mRNA serves to modulate splicing, either by masking a binding site for a native protein that would otherwise modulate splicing and/or by altering the structure of the targeted RNA. In some embodiments, the target RNA is target pre-mRNA (e.g., LIN28B gene pre-mRNA).

An antisense oligomer having a sufficient sequence complementarity to a target RNA sequence to modulate splicing of the target RNA means that the antisense oligomer has a sequence sufficient to trigger the masking of a binding site for a native protein that would otherwise modulate splicing and/or alters the three-dimensional structure of the targeted RNA.

Selected antisense oligomers can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the antisense oligomer is selected from the group comprising the sequences set forth in Table 1. Preferably, the antisense oligomer is selected from the group comprising the sequences in SEQ ID NOs: 1-43, more preferably SEQ ID NOs: 2, 4, 5 and 6. Most preferably, the antisense oligomer is SEQ ID NO: 2.

In certain embodiments, the degree of complementarity between the target sequence and antisense oligomer is sufficient to form a stable duplex. The region of complementarity of the antisense oligomers with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An antisense oligomer of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligomer (PMO) antisense oligomers, an optimum balance of binding stability and uptake generally occurs at lengths of 18-25 bases. Included are antisense oligomers (e.g., CPPMOs, PPMOs, PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, antisense oligomers may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing of the target pre-mRNA is modulated.

The stability of the duplex formed between an antisense oligomer and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included.

Additional examples of variants include antisense oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOs: 1-43, more preferably SEQ ID NOs: 2, 4, 5 and 6. Most preferably, the antisense oligomer is SEQ ID NO: 2 or the sequences provided in Table 1.

More specifically, there is provided an antisense oligomer capable of binding to a selected target site to modify pre-mRNA splicing in a LIN28B gene transcript or part thereof. The antisense oligomer is preferably selected from those provided in Table 1 or SEQ ID NOs: 1-43, more preferably SEQ ID NOs: 2, 4, 5 and 6. Most preferably, the antisense oligomer is SEQ ID NO: 2.

The modification of pre-mRNA splicing preferably induces “skipping”, or the removal of one or more exons or introns of the mRNA and/or terminal intron retention. The resultant protein may be of a shorter length when compared to the parent full-length LIN28B protein due to either internal truncation or premature termination or may be longer due to terminal intron retention. These LIN28B proteins may be termed isoforms of the unmodified LIN28B protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (eg. a nonsense sequence) due to a frame-shift. Alternatively, the exon skipping may induce the production of a prematurely terminated protein due to a disruption of the reading frame and presence of a premature termination of translation. Additionally, the antisense oligomer may produce an artificially lengthened protein, due to in-frame terminal intron retention.

Exclusion of exon-2 leads to the induction of 5 premature termination codons in exon-3 and exon-4 respectively. Therefore, exclusion of exon-2 will lead to a reduction or elimination of transcription of the LIN28B protein, due to premature termination of the RNA.

The removal of one or more exons may further lead to misfolding of the LIN28B protein and a reduction in the ability of the protein to be successfully transported through the membrane.

The antisense oligomer induced exon skipping of the present invention need not completely or even substantially ablate the function of the LIN28B protein, but may result in a reduced or compromised functionality of the LIN28B protein. However, preferably the exon skipping process results in complete or substantially complete ablation of the functionality of the LIN28B protein.

The skipping process of the present invention, using antisense oligomers, may skip an individual exon, or may result in skipping two or more exons at once.

The antisense oligomers of the invention may be a combination of two or more antisense oligomers capable of binding to a selected target to induce exon exclusion in a LIN28B gene transcript. The combination may be a cocktail of two or more antisense oligomers and/or a construct comprising two or more or two or more antisense oligomers joined together.

TABLE 1
ASO designs targeting LIN28B. Sequences are either in full DNA, 2′-OME PS,
2′-OMOE PS or PMO chemistries. ‘T’ nucleotide bases are replaced with
‘U’ nucleotide bases in the case of 2′-OMe PS modifications. PS =
phosphorothioate; 2′-OMe = 2′-O-Methyl RNA; 2′-OMOE = 2′-Methoxy
ethoxy RNA; PMO = Phosphorodiamidate morpholino oligomer.

| Sequence | Targeting | | |
|---|---|---|---|
| number | exon | Coordinate | Sequence (ASO) |
| 1 | Exon-2 | LIN28B 1E2A (−10 +15) | CTT TGC TAG CCC CGC CTG AGA AGG A |
| 2 | Exon-2 | LIN28B 1E2A (+10 +34) | CCG GGC TCT TCT CCA CCA CCT TTG C |
| | | (Linexol-2) | |
| 3 | Exon-2 | LIN28B 1E2A (+37 +61) | TCC TCC TCT GCC GGC TCC GGC AGC T |
| 4 | Exon-2 | LIN28B 1E2A (+45 +69) | CCT GGG ATT CCT CCT CTG CCG GCT C |
| 5 | Exon-2 | LIN28B 1E2A (+69 +93) | TAC AGT GGC CAG TTC CGC GCA AAA C |
| 6 | Exon-2 | LIN28B 1E2A (+142 +166) | GGA ATA TCC AAG GGG CTT CCC TCT C |
| 7 | Exon-3 | LIN28B 1E3A (−10 +15) | CAT GAA TAG TTT GCT CTG TAA AGA G |
| 8 | Exon-3 | LIN28B 1E3A (+15 +39) | TTC TTT TAG GCT TCT AAA TCC TTC C |
| 9 | Exon-3 | LIN28B 1E3A (+40 +64) | TAA ATG TGA ATT CCA CTG GTT CTC C |
| 10 | Exon-3 | LIN28B 1E3A (+65 +89) | GAC TCA AGG CCT TTG GAA GAT TTT T |
| 11 | Exon-3 | LIN28B 1E3A (+90 +114) | CCC ACC AGG TCC TGT TAC CCG TAT T |
| 12 | Exon-3 | LIN28B 1E3A (+115 +139) | TTC TTT CAC TTC CTA AAC AGG GGC T |
| 13 | Exon-3 | LIN28B 1E3A (+140 +164) | TTC TGT AGT GTC TTC CCT TTG GGT C |
| 14 | Exon-3 | LIN28B 1E3D (−4 +21) | TTA CCT ATC TCC CTT TGG TTT TCT T |
| 15 | Exon-1 | LIN28B 1E1A (+1 +25) | GGT CTA ATG TGC TTT CCT TCT TTT G |
| 16 | Exon-1 | LIN28B 1E1A (+17 +41) | CAA ATT TAG CTC GCA TGG TCT AAT G |
| 17 | Exon-1 | LIN28B 1E1A (+39 +63) | ACA TCT TGA TTT TGT GCG ATC ATA A |
| 18 | Exon-1 | LIN28B 1E1A (+64 +88) | CGG AGTGAT CTT CTG CAT CAA TCT A |
| 19 | Exon-1 | LIN28B 1E1A (+89 +113) | TGA GAT GAA AAC TTT CCC TTT GGA A |
| 20 | Exon-1 | LIN28B 1E1A (+114 +138) | CCA CGG GCC CTC AGC TCC AAA CTC G |
| 21 | Exon-1 | LIN28B 1E1A (+130 +154) | CTT CGGCCA TGT TGC CCC ACG GGC C |
| 22 | Exon-4 | LIN28B 1E4A (+11 +35) | TAG CAT GAT GAT CAA GGC CAC CAC A |
| 23 | Exon-4 | LIN28B 1E4A (+28 +52) | AGG TAG ACT ACA TTC CTT AGC ATG A |
| 24 | Exon-4 | LIN28B 1E4A (+200 +224) | ATG TACAGC CAT GCC CGC CTC CCA C |
| 25 | Exon-4 | LIN28B 1E4A (+225 +249) | CTA GCC TCC TGA GGA AAC GGT GGT G |
| 26 | Exon-4 | LIN28B 1E4A (+580 +604) | TCA CAC ATG CAC ATA TTG ACA GAC A |
| 27 | Exon-4 | LIN28B 1E4A (+605 +629) | CAC ACA GAC TCA GGC TCT CTC CCT C |
| 28 | Exon-4 | LIN28B 1E4A (+626 +650) | ATA TAA AAA TCC TCA TGT ACA CAC A |
| 29 | Exon-4 | LIN28B 1E4A (+961 +985) | ATT ACT ACT CCA GAG ATT TCA AGA A |
| 30 | Exon-4 | LIN28B 1E4A (+986 +1010) | CTG CCT TCA AAA AAG GGG GAA AAA A |
| 31 | Exon-4 | LIN28B 1E4A (+996 +1020) | AGT TAA GGT ACT GCC TTC AAA AAA G |
| 32 | Exon-4 | LIN28B 1E4A (+2560 +2584) | TCA AAT TGC ACC CAT AAA GAC CAT A |
| 33 | Exon-4 | LIN28B 1E4A (+2585 +2609) | GTC TGG TAA GAT GAT GAA AAG GAT T |
| 34 | Exon-4 | LIN28B 1E4A (+2596 +2620) | CTC TTA GTT TAG TCT GGT AAG ATG A |
| 35 | Exon-4 | LIN28B 1E4A (+2670 +2694) | TTA TGT AAT AGT GAT AAT ATT CTC A |
| 36 | Exon-4 | LIN28B 1E4A (+2695 +2719) | AGC AAA GTT CTT TGT CCT GAG TAT G |
| 37 | Exon-4 | LIN28B 1E4A (+4160 +4184) | CAT TAA GAC ATC TAG AAA ATT AAA G |
| 38 | Exon-4 | LIN28B 1E4A (+4185 +4209) | GTT CTT CTC TAA AAC AAA TAA ATC T |
| 39 | Exon-4 | LIN28B 1E4A (+4206 +4230) | GTT TGA TGC TTT TAA CAA GAT GTT C |
| 40 | Exon-4 | LIN28B 1E4A (+4430 +4454) | CAT CAT TTT CAA GCT GGT AAC TCT C |
| 41 | Exon-4 | LIN28B 1E4A (+4455 +4479) | ATG TGA TTC AAG AGG TAG TCA ACA C |
| 42 | Exon-4 | LIN28B 1E4A (+4480 +4504) | GGT GGT AGG TGC CAG TGG TTG ATA G |
| 43 | Exon-4 | LIN28B 1E4A (+4495 +4520) | TAA TTG AAG CCA GCT TGG TGG TAG G |

There is also provided a method for manipulating splicing in a LIN28B gene transcript, the method including the step of:

a) providing one or more of the antisense oligomers as described herein and allowing the oligomer(s) to bind to a target nucleic acid site.

According to yet another aspect of the invention, there is provided a splice manipulation target nucleic acid sequence for LIN28B comprising the DNA equivalents of the nucleic acid sequences selected from Table 1 or the group consisting of SEQ ID NOs: 1-43, more preferably SEQ ID NOs: 2, 4, 5 and 6, and sequences complementary thereto. Most preferably, the antisense oligomer is SEQ ID NO: 2, and sequences complementary thereto.

Designing antisense oligomers to completely mask consensus splice sites may not necessarily generate a change in splicing of the targeted exon. Furthermore, the inventors have discovered that size or length of the antisense oligomer itself is not always a primary factor when designing antisense oligomers. With some targets such as IGTA4 exon-3, antisense oligomers as short as 20 bases were able to induce some exon skipping, in certain cases more efficiently than other longer (eg 25 bases) oligomers directed to the same exon.

The inventors have also discovered that there does not appear to be any standard motif that can be blocked or masked by antisense oligomers to redirect splicing. It has been found that antisense oligomers must be designed and their individual efficacy evaluated empirically.

More specifically, the antisense oligomer may be selected from those set forth in Table 1. The sequences are preferably selected from the group consisting of any one or more of any one or more of SEQ ID NOs: 1-43, more preferably SEQ ID NOs: 2, 4, 5 and 6. Most preferably, the antisense oligomer is SEQ ID NO: 2, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a LIN28B gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an antisense oligomer need not be 100% complementary to that of its target sequence to be specifically hybridisable. An antisense oligomer is specifically hybridisable when binding of the compound to the target DNA or RNA molecule interferes with the normal function of the target DNA or RNA product, and there is a sufficient degree of complementarity to avoid non-specific binding of the antisense oligomer to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the antisense oligomers of the present invention may include oligomers that selectively hybridise to the sequences provided in Table 1, or SEQ ID NOs: 1-43, more preferably SEQ ID NOs: 2, 4, 5 and 6. Most preferably, the antisense oligomer is SEQ ID NO: 2.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligomers may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the antisense oligomer sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an antisense oligomer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligomers set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The antisense oligomer of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or antisense oligomers or compositions to produce or cause a greater physiological response (i.e., downstream effects) in a cell or a subject relative to the response caused by either no antisense oligomer or a control compound. The terms “decreasing” or “decrease” refer generally to the ability of one or antisense oligomers or compositions to produce or cause a reduced physiological response (i.e., downstream effects) in a cell or a subject relative to the response caused by either no antisense oligomer or a control compound.

Relevant physiological or cellular responses (in vivo or in vitro) will be apparent to persons skilled in the art, and may include increases in the exclusion of specific exons in a LIN28B-coding pre-mRNA, decreases in the amount of LIN28B-coding pre-mRNA or decreases in the expression of functional LIN28B protein in a cell, tissue, or subject in need thereof. An “decreased” or “reduced” amount is typically a statistically significant amount, and may include a decrease that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) less than the amount produced when no antisense oligomer is present (the absence of an agent) or a control compound is used.

The term “reduce” or “inhibit” may relate generally to the ability of one or more antisense oligomers or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a cancer, particularly a solid tumour cancer.

A “decrease” in a response may be statistically significant as compared to the response produced by no antisense oligomer or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligomer may vary, as long as it is capable of binding selectively to the intended location within the pre-mRNA molecule. The length of such sequences can be determined in accordance with selection procedures described herein. Generally, the antisense oligomer will be from about 10 nucleotides in length, up to about 50 nucleotides in length. It will be appreciated, however, that any length of nucleotides within this range may be used in the method. Preferably, the length of the antisense oligomer is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “antisense oligomer” (ASO) refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The terms “antisense oligomer”, “antisense oligonucleotide”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, among other antisense agents known in the art.

Included are non-naturally-occurring antisense oligomers, or “oligonucleotide analogs”, including antisense oligomers or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing antisense oligomers is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA during duplex formation with the antisense oligomers, the antisense oligomers used in the method may be adapted to minimise or prevent cleavage by endogenous RNase H. This property is highly preferred, as the treatment of the RNA with the unmethylated oligomers, either intracellular or in crude extracts that contain RNase H, leads to degradation of the pre-mRNA:antisense oligomer duplexes. Any form of modified antisense oligomers that is capable of by-passing or not inducing such degradation may be used in the present method. The nuclease resistance may be achieved by modifying the antisense oligomers of the invention so that it comprises partially unsaturated aliphatic hydrocarbon chain and one or more polar or charged groups including carboxylic acid groups, ester groups, and alcohol groups.

Antisense oligomers that do not activate RNase H can be made in accordance with known techniques (see, e.g., U.S. Pat. No. 5,149,797). Such antisense oligomers, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous antisense oligomers that do not activate RNase H are available. For example, such antisense oligomers may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such antisense oligomers are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

An example of antisense oligomers which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo- counterparts. Alternatively, the nuclease resistant antisense oligomers of the invention may have at least one of the last 3′-terminus nucleotides fluoridated. Still alternatively, the nuclease resistant antisense oligomers of the invention have phosphorothioate bonds linking between at least two of the last 3-terminus nucleotide bases, preferably having phosphorothioate bonds linking between the last four 3′-terminal nucleotide bases.

Increased splice-switching may also be achieved with alternative oligonucleotide chemistry. For example, the antisense oligomer may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′-O-Methyl-modified oligomer (2′-OMe); 2′-O-methoxy ethyl (2′-MOE); 2′-fluoro, 2′-fluroarabino (FANA); unlocked nucleic acid (UNA); hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping antisense oligomers. Antisense oligomer-induced splice modification of the human LIN28B gene transcripts have generally used either oligoribonucleotides, PNAs, 2′-OMe or MOE modified bases on a phosphorothioate backbone. Although 2′-OMe AOs are used for oligo design, due to their efficient uptake in vitro when delivered as cationic lipoplexes, these compounds are susceptible to nuclease degradation and are not considered ideal for in vivo or clinical applications. When alternative chemistries are used to generate the antisense oligomers of the present invention, the uracil (U) of the sequences provided herein may be replaced by a thymine (T).

While the antisense oligomers described above are a preferred form of the antisense oligomers of the present invention, the present invention includes other oligomeric antisense molecules, including but not limited to oligomer mimetics such as are described below.

Specific examples of preferred antisense oligomers useful in this invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be antisense oligomers.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to an RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H- phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligomer. The present invention also includes antisense oligomers that are chimeric compounds. “Chimeric” antisense oligomers or “chimeras,” in the context of this invention, are antisense oligomers, particularly oligomers, which contain two or more chemically distinct regions, each made up of at least one monomer unit, i.e., a nucleotide in the case of an oligomer compound. These oligomers typically contain at least one region wherein the oligomer is modified so as to confer upon the oligomer or antisense oligomer increased resistance to nuclease degradation, increased cellular uptake, and an additional region for increased binding affinity for the target nucleic acid.

The activity of antisense oligomers and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The present invention provides antisense oligomer induced splice-switching of the LIN28B gene transcript, clinically relevant oligomer chemistries and delivery systems to direct LIN28B splice manipulation to therapeutic levels. Substantial decreases in the amount of full length LIN28B mRNA, and hence LIN28B protein from LIN28B gene transcription, are achieved by:
1) oligomer refinement in vitro using fibroblast cell lines, through experimental assessment of (i) intronic -enhancer target motifs, (ii) antisense oligomer length and development of oligomer cocktails, (iii) choice of chemistry, and (iv) the addition of cell-penetrating peptides (CPP) to enhance oligomer delivery; and
2) detailed evaluation of a novel approach to generate LIN28B transcripts with one or more missing exons.

As such, it is demonstrated herein that processing of LIN28B pre-mRNA can be manipulated with specific antisense oligomers. In this way functionally significant decreases in the amount of LIN28B protein can be obtained, thereby reducing the severe pathology associated with cancers, particularly solid tumour cancers.

The antisense oligomers used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligomers of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules etc.

The antisense oligomers may be formulated for oral, topical, parenteral or other delivery, particularly formulations for injectable delivery. The formulations may be formulated for assisting in uptake, distribution and/or absorption at the site of delivery or activity. Preferably the antisense oligomers of the present invention are formulated for delivered via injection.

### Method of Treatment

According to a still further aspect of the invention, there is provided one or more antisense oligomers as described herein for use in an antisense oligomer-based therapy. Preferably, the therapy is for a cancer related to LIN28B expression, more preferably a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer or brain cancer. The cancer to be treated is preferably liver cancer or brain cancer.

More specifically, the antisense oligomer may be selected from Table 1, or the group consisting of any one or more of SEQ ID NOs: 1-43, more preferably SEQ ID NOs: 2, 4, 5 and 6. Most preferably, the antisense oligomer is SEQ ID NO: 2, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a LIN28B gene transcript.

The invention extends also to a combination of two or more antisense oligomers capable of binding to a selected target to induce exon exclusion in a LIN28B gene transcript. The combination may be a cocktail of two or more antisense oligomers, a construct comprising two or more or two or more antisense oligomers joined together for use in an antisense oligomer-based therapy.

There is therefore provided a method to treat or ameliorate the effects of a cancer associated with LIN28B expression, comprising the step of:

a) administering to the subject an effective amount of one or more antisense oligomers or pharmaceutical composition comprising one or more antisense oligomers as described herein.

Preferably, the therapy is for a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer.

Therefore, the invention provides a method to treat or ameliorate the effects of a solid tumour cancer associated with LIN28B expression, comprising the step of:

a) administering to the subject an effective amount of one or more antisense oligomers or pharmaceutical composition comprising one or more antisense oligomers as described herein.

Preferably, the therapy is used to reduce the levels of functional LIN28B protein via an exon skipping strategy. The reduction in levels of LIN28B is preferably achieved by reducing the transcripts level through modifying pre-mRNA splicing in the LIN28B gene transcript or part thereof.

The reduction in LIN28B will preferably lead to a reduction in the quantity, duration or severity of the symptoms of a cancer associated with LIN28B expression, such as a solid tumour cancer. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The subject with the disease associated with LIN28B expression may be a mammal, including a human.

The antisense oligomers of the present invention may also be used in conjunction with alternative therapies, such as drug therapies including chemotherapy, surgery or radiotherapy.

The present invention therefore provides a method of treating, preventing or ameliorating the effects of a cancer associated with LIN28B expression, wherein the antisense oligomers of the present invention and administered sequentially or concurrently with another alternative therapy associated with treating or ameliorating the effects of a cancer associated with LIN28B expression. Preferably, the therapy is for a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer.

### Delivery

The antisense oligomers of the present invention also can be used as a prophylactic or therapeutic, which may be utilised for the purpose of treatment of a disease. Accordingly, in one embodiment the present invention provides antisense oligomers that bind to a selected target in the LIN28B pre-mRNA to induce efficient and consistent exon skipping as described herein, in a therapeutically effective amount, admixed with a pharmaceutically acceptable carrier, diluent, or excipient.

There is also provided a pharmaceutical, prophylactic, or therapeutic composition to treat, prevent or ameliorate the effects of a disease related to LIN28B expression in a subject, the composition comprising:

a) one or more antisense oligomers as described herein; and

b) one or more pharmaceutically acceptable carriers and/or diluents.

Preferably, the antisense oligomer of the present invention is delivered via a parenteral route. For example, the antisense oligomer may be injected into the tumour, or administered via intravenous, intramuscular or subcutaneous injection for a more systemic effect.

The antisense oligomer may be administered at regular intervals for a short time period, e.g., daily for two weeks or less. However, in many cases the oligomer is administered intermittently over a longer period of time. Administration may be followed by, or concurrent with, administration of chemotherapy, surgery or radiotherapy or other therapeutic treatment. The treatment regimen may be adjusted (dose, frequency, route, etc.) as indicated, based on the results of immunoassays, other biochemical tests and physiological examination of the subject under treatment.

Dosing may be dependent on severity and responsiveness of the disease state to be treated, with the course of treatment lasting from several days to several months, or until a cure is effected or a diminution of the disease state is achieved. Alternatively, dosing may be titrated against disease progression rate. A baseline progression is established. Then the progression rate after an initial once off dose is monitored to check that there is a reduction in the rate. Preferably, there is no progression after dosing. Preferably, re-dosing is only necessary if progression rate is unchanged. Successful treatment preferably results in no further progression of the disease or even remission. Optimal dosing schedules can be calculated from measurements of drug accumulation in the body of the subject. Persons of ordinary skill can easily determine optimum dosages, dosing methodologies and repetition rates.

Optimum dosages may vary depending on the relative potency of individual oligomers, and can generally be estimated based on EC50s found to be effective in in vitro and in vivo animal models.

An effective in vivo treatment regimen using the antisense oligomers of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligomers of the invention may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

Intranuclear oligomer delivery is a major challenge for antisense oligomers. Different cell-penetrating peptides (CPP) localize PMOs to varying degrees in different conditions and cell lines, and novel CPPs have been evaluated by the inventors for their ability to deliver PMOs to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 2010/0016215, which is incorporated by reference in its entirety.

The present invention therefore provides antisense oligomers of the present invention in combination with cell-penetrating peptides for manufacturing therapeutic pharmaceutical compositions.

### Excipients

The antisense oligomers of the present invention are preferably delivered in a pharmaceutically acceptable composition. The composition may comprise about 1 nM to 1000 μM of each of the desired antisense oligomer(s) of the invention.

The present invention further provides one or more antisense oligomers adapted to aid in the prophylactic or therapeutic treatment or amelioration of the effects of a cancer associated with LIN28B expression related disease or pathology in a form suitable for delivery to a subject.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Remington: The Science and Practice of Pharmacy, 22nd Ed., Pharmaceutical Press, PA (2013).

In a more specific form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Remington: The Science and Practice of Pharmacy, 22nd Ed., Pharmaceutical Press, PA (2013). The compositions may be prepared in liquid form, or may be in dried powder, such as a lyophilised form.

It will be appreciated that pharmaceutical compositions provided according to the present invention may be administered by any means known in the art. The pharmaceutical compositions for administration are administered by injection, orally, topically or by the pulmonary or nasal route. For example, the antisense oligomers may be delivered by intravenous, intra-arterial, intraperitoneal, intramuscular or subcutaneous routes of administration. The appropriate route may be determined by one of skill in the art, as appropriate to the condition of the subject under treatment. Preferably, the antisense oligomers are parenterally delivered, for example via injection into the solid tumour or via intravenous, subcutaneous or intramuscular administration.

The delivery of a therapeutically useful amount of antisense oligomers may be achieved by methods previously published. For example, delivery of the antisense oligomer may be via a composition comprising an admixture of the antisense oligomer and an effective amount of a block copolymer. An example of this method is described in US patent application US20040248833. Other methods of delivery of antisense oligomers to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

It may be desirable to deliver the antisense oligomer in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligomer of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

As known in the art, antisense oligomers may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomer may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The antisense oligomers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be via topical (including ophthalmic and mucous membranes, as well as rectal delivery), pulmonary (e.g., by inhalation or insufflation of powders or aerosols, including by nebulizer, intratracheal, intranasal, epidermal and transdermal), oral or parenteral routes. Parenteral administration includes intravenous, intra-arterial, subcutaneous, intraperitoneal, intraocular or intramuscular injection or infusion; or intracranial, e.g., intrathecal or intraventricular, administration. Oligomers with at least one 2′-O-methoxyethyl modification are believed to be particularly useful for parenteral administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

### Swiss-Style

According to another aspect of the invention there is provided the use of one or more antisense oligomers as described herein in the manufacture of a medicament for the treatment or amelioration of the effects of a cancer associated with LIN28B expression.

The invention also provides for the use of purified and isolated antisense oligomers as described herein, for the manufacture of a medicament for treatment or amelioration of the effects of a cancer associated with LIN28B expression.

Preferably, the therapy is for a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer.

The invention extends, according to a still further aspect thereof, to cDNA or cloned copies of the antisense oligomer sequences of the invention, as well as to vectors containing the antisense oligomer sequences of the invention. The invention extends further also to cells containing such sequences and/or vectors.

### Kits

There is also provided a kit to treat or ameliorate the effects of a cancer associated with LIN28B expression in a subject, which kit comprises at least an antisense oligomer as described herein and combinations or cocktails thereof, packaged in a suitable container, together with instructions for its use.

In a preferred embodiment, the kits will contain at least one antisense oligomer as described herein or as shown in Table 1, or SEQ ID NOs: 1-43, more preferably SEQ ID NOs: 2, 4, 5 and 6, most preferably SEQ ID NO: 2 or a cocktail of antisense oligomers, as described herein. The kits may also contain peripheral reagents such as buffers, stabilizers, etc.

There is therefore provided a kit to treat or ameliorate the effects of a cancer associated with LIN28B expression in a subject, which kit comprises at least an antisense oligomer described herein or as shown in Table 1 and combinations or cocktails thereof, packaged in a suitable container, together with instructions for its use.

There is also provided a kit to treat or ameliorate the effects of a cancer associated with LIN28B expression in a subject, which kit comprises at least an antisense oligomer selected from the group consisting of any one or more of SEQ ID NOs: 1-43, more preferably SEQ ID NOs: 2, 4, 5 and 6, most preferably SEQ ID NO: 2, and combinations or cocktails thereof, packaged in a suitable container, together with instructions for its use.

Preferably, the therapy is for a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

In an embodiment, the kit of the present invention comprises a composition comprising a therapeutically effective amount of an antisense oligomer capable of binding to a selected target on a LIN28B gene transcript to modify pre-mRNA splicing in a LIN28B gene transcript or part thereof. In an alternative embodiment, the formulation is in pre-measured, pre-mixed and/or pre-packaged. Preferably, the kit is for parenteral administration and the solution is sterile.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the formulations of the present invention. In one embodiment, the instructions indicate that the formulation of the present invention is suitable for the treatment of a solid tumour cancer related to LIN28B expression. More preferably, the solid tumour cancer is chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer. The cancer to be treated is preferably liver cancer or brain cancer. Such instructions may also include instructions on dosage, as well as instructions for administration.

The antisense oligomers and suitable excipients can be packaged individually so to allow a practitioner or user to formulate the components into a pharmaceutically acceptable composition as needed. Alternatively, the antisense oligomers and suitable excipients can be packaged together, thereby requiring de minimus formulation by the practitioner or user. In any event, the packaging should maintain chemical, physical, and aesthetic integrity of the active ingredients.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

Sequence identity numbers (“SEQ ID NO:”) containing nucleotide and amino acid sequence information included in this specification are collected at the end of the description and have been prepared using the program PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense oligomer nomenclature system was proposed and published to distinguish between the different antisense oligomers (see Mann et al., (2002) J Gen Med 4, 644-654). This nomenclature became especially relevant when testing several slightly different antisense oligomers, all directed at the same target region, as shown below:

H #A/D (x:y)

the first letter designates the species (e.g. H: human, M: murine)

“#” designates target exon number

“A/D” indicates acceptor or donor splice site at the beginning and end of the exon, respectively

(x y) represents the annealing coordinates where “−” or “+” indicate intronic or exonic sequences respectively. As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense oligomer. Entirely exonic annealing coordinates that would be represented by A(+65+85), that is the site between the 65th and 85th nucleotide, inclusive, from the start of that exon.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Expression Analysis of LIN28B in Various Cancer Cells and Normal Cells

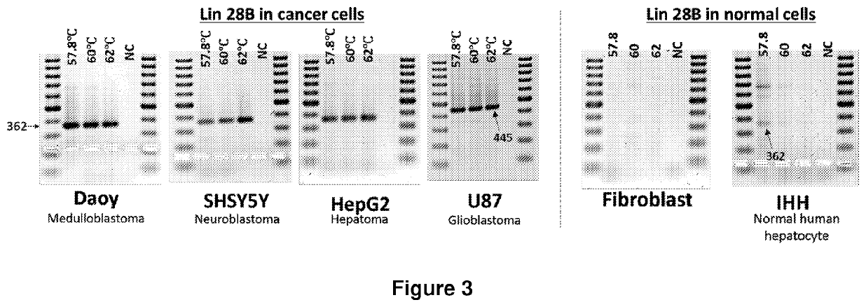

LIN28B expression was analysed in liver cancer (HepG2), medulloblastoma (DAOY), neuroblastoma (SHSY5Y), and glioblastoma cells (U87MG), which showed a high expression of LIN28B (FIG. 3) using primer pairs that gives the product of either 362 bp or 445 bp (Table 2). Notably, when performed in normal cells such as human liver hepatocytes (IHH cells), LIN28B was found to be negative in most cases, very weak expression could be found only under certain condition. In human fibroblasts (primary), LIN28B expression was found to be completely negative (FIG. 3).

TABLE 2
Forward and reverse primer designs for LIN28B analysis

| SEQ ID | | | Product |
|---|---|---|---|
| NO | Forward Primer | Reverse Primer | sizes |
| 44, 45 | LIN28B_A_Ex1F (44) | LIN28B_A_Ex3R (45) | 362 bp |
| | TCA CGA GTT TGG AGC TGA GG | TTC CTA AAC AGG GGC TCC CA | |
| 46, 47 | LIN28B_B_Ex1F (46) | LIN28B_B_Ex3R (47) | 445 bp |
| | GCA CAT TAG ACC ATG CGA GC | CTC CCA CCA GGT CCT GTT AC | |

### Example 2

### Antisense Oligonucleotide Designs

ASOs targeting exon-2 were designed for exon skipping, translational blocking or RNase H-based inhibition of LIN28B (Table 1). The ASOs were initially synthesised and tested with fully 2′-OMePS chemistry, but later on the best performing ASO was synthesised with fully 2′-O-MOE-PS, 7-11-7 2′-O-MOE gapmer and fully PMO chemistries.

### Example 3

### Evaluation of LIN28B Targeting AOs in Cancer Cells

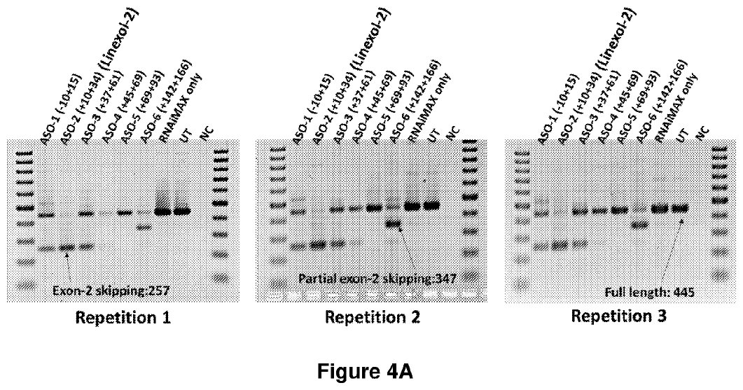

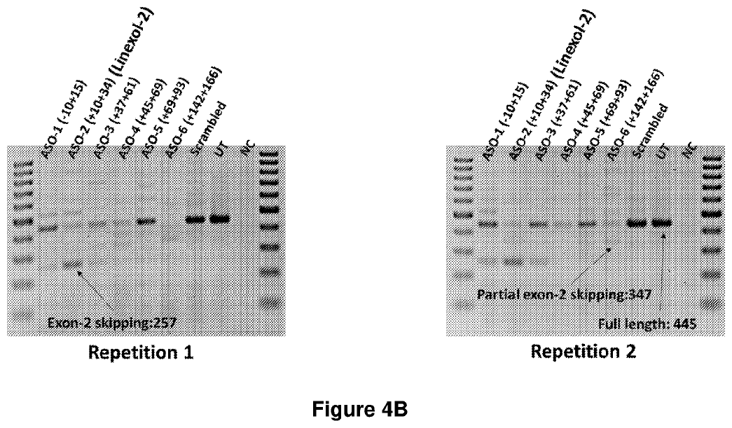

First, we have tested the efficacies of all synthesised exon-2 targeting 2′-OMe-PS ASOs in HepG2 liver cancer cells at 400 nM concentration. Remarkably, the experiments revealed that all exon-2 targeting ASOs were able to inhibit LIN28B and except ASO-5: LIN28B 1E2A (+69+93), all exon-2 targeting ASOs also induced exon-2 skipping (product at 257 bp) or partial exon-2 skipping (product at 347 bp) at various yields (FIG. 4A). ASO LIN28B 1E2A (+10+34) was found to be the most efficient to induce exon-2 skipping. ASOs LIN28B 1E2A (+45+69), LIN28B 1E2A (+69+93) and LIN28B 1E2A (+142+166) were found to inhibit LIN28B RNA efficiently. Similar results were also observed in U87MG glioblastoma cells (FIG. 4B).

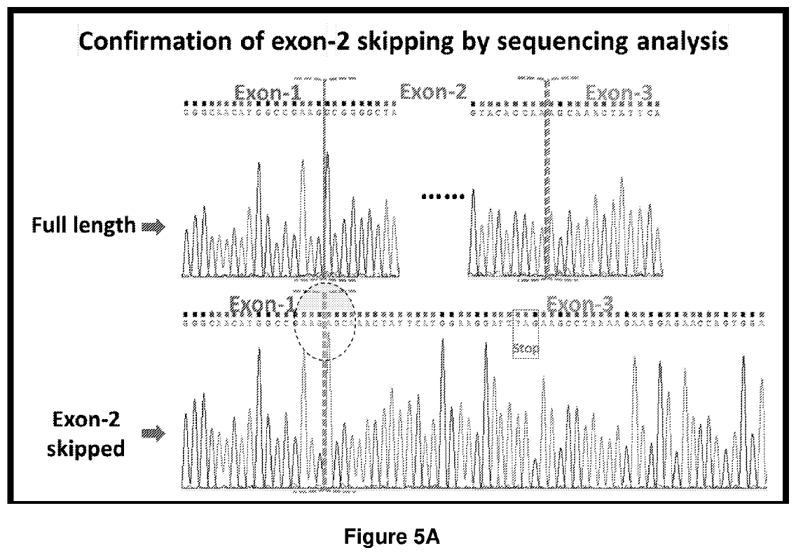

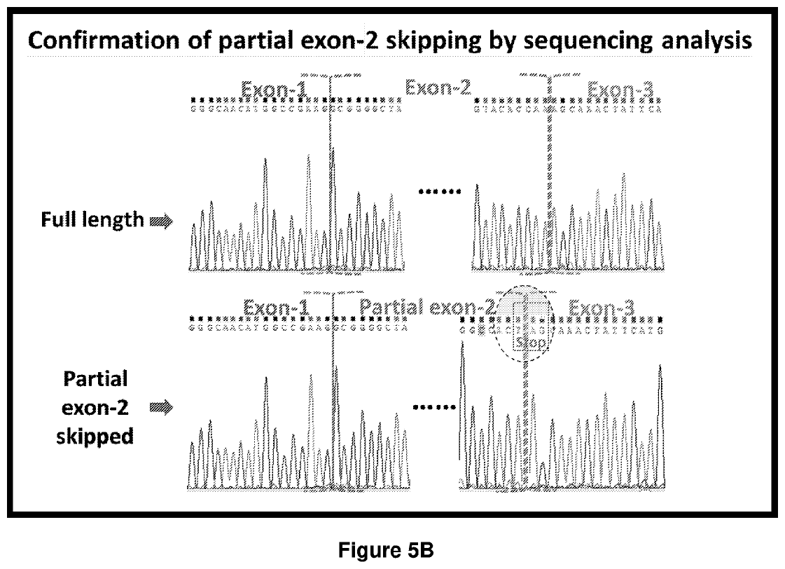

Exon-2 skipping was confirmed by verifying the skipped product of 257 bp by performing Sanger sequencing analysis (FIG. 5A). Partial exon-2 skipping was confirmed by verifying the skipped product of 347 bp by performing Sanger sequencing analysis (FIG. 5B).

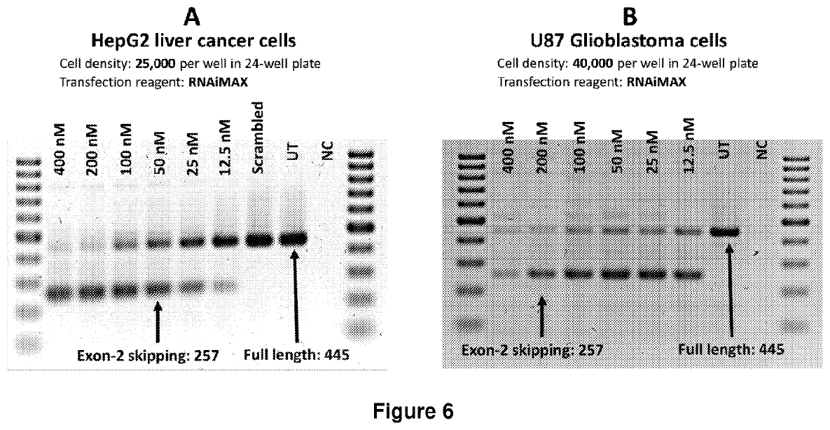

As ASO-2: LIN28B 1E2A (+10+34) was found to be very efficient to induce exon-2 skipping, dose dependency was then initiated in both liver and brain cancer cells. This experiment was performed using 6 concentrations such as 12.5, 25, 50, 100, 200, and 400 nM respectively over 24 h after transfection. Our results showed that the ASO-2: LIN28B 1E2A (+10+34) efficiently induced exon-2 skipping in a dose-dependent manner, and at 100 nM concentration, almost full exon-2 skipping was observed in both Liver cancer (FIG. 6A) and in brain cancer cells (FIG. 6B).

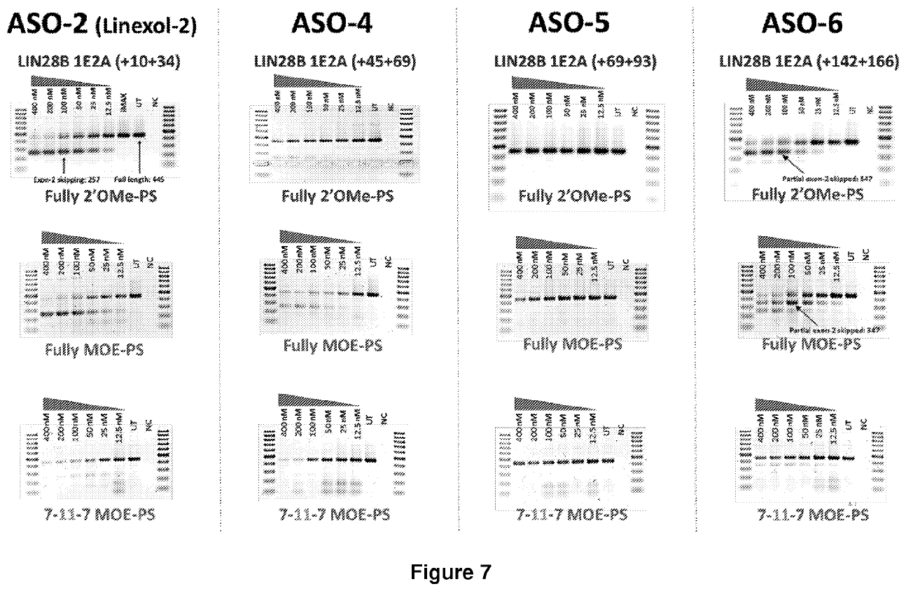

LIN28B RNA inhibition efficacy of different chemistries of ASO-2, ASO-4, ASO-5, and ASO-6 were evaluated by dose dependency test in liver cancer HepG2 cells (Table 3A-3D, FIG. 7). Based on the results, ASO-2: LIN28B 1E2A (+10+34) was the best sequence out of the four sequences, and fully 2′-MOE-PS was the best design out of the three designs in the inhibition or knockdown efficacy of full length LIN28B transcript.

TABLE 3A
LIN28B RNA inhibition efficacy of different chemistries of ASO-2, evaluated by dose dependency test in liver cancer HepG2 cells
A. ASO-2 (Linexol-2) LIN28B 1E2A (+10 +34)

| A1. 2′-OMe-PS | | | | | | A2. 2′-MOE-PS | | | | | | A3. 7-11-7 MOE gapmer | | | | | |
|---|---|---|---|---|---|---|---|---|---|---|---|---|---|---|---|---|---|
| | | | | | Full | | | | | | Full | | | | | | Full |
| | | | | | length | | | | | | length | | | | | | length |
| | | | | | knock- | | | | | | knock- | | | | | | knock- |
| | | | | | down | | | | | | down | | | | | | down |
| | | | | | effic- | | | | | | effic- | | | | | | effic- |
| ASO | | Exon skipping | | | iency | ASO | | Exon skipping | | | iency | ASO | | Exon skipping | | | iency |
| treat- | | efficiency | | | (com- | treat- | | efficiency | | | (com- | treat- | | efficiency | | | (com- |
| ment | | | | Partial | pared | ment | | | | Partial | pared | ment | | | | Partial | pared |
| concen- | | Full | Exon-2 | exon-2 | to un- | concen- | | Full | Exon-2 | exon-2 | to un- | concen- | | Full | Exon-2 | exon-2 | to un- |
| tration | | length | skipped | skipped | treated) | tration | | length | skipped | skipped | treated) | tration | | length | skipped | skipped | treated) |
| 400 | nM | 10% | 90% | 0% | 93% | 400 | nM | 7% | 93% | 0% | 95% | 400 | nM | 88% | 12% | 0% | 91% |
| 200 | nM | 15% | 85% | 0% | 87% | 200 | nM | 15% | 85% | 0% | 86% | 200 | nM | 89% | 11% | 0% | 87% |
| 100 | nM | 33% | 65% | 0% | 63% | 100 | nM | 29% | 71% | 0% | 72% | 100 | nM | 70% | 30% | 0% | 85% |
| 50 | nM | 44% | 56% | 0% | 54% | 50 | nM | 47% | 53% | 0% | 70% | 50 | nM | 73% | 27% | 0% | 75% |
| 25 | nM | 57% | 43% | 0% | 44% | 25 | nM | 67% | 33% | 0% | 61% | 25 | nM | 89% | 11% | 0% | 55% |
| 12.5 | nM | 75% | 25% | 0% | 31% | 12.5 | nM | 85% | 15% | 0% | 55% | 12.5 | nM | 91% | 9% | 0% | 44% |

TABLE 3B
LIN28B RNA inhibition efficacy of different chemistries of ASO-4, evaluated by dose dependency test in liver cancer HepG2 cells
B. ASO-4 LIN28B 1E2A (+45 +6S)

| B1. 2′-OMe-PS | | | | | | B2. 2′-MOE-PS | | | | | | B3. 7-11-7 MOE gapmer | | | | | |
|---|---|---|---|---|---|---|---|---|---|---|---|---|---|---|---|---|---|
| | | | | | Full | | | | | | Full | | | | | | Full |
| | | | | | length | | | | | | length | | | | | | length |
| | | | | | knock- | | | | | | knock- | | | | | | knock- |
| | | | | | down | | | | | | down | | | | | | down |
| | | | | | effic- | | | | | | effic- | | | | | | effic- |
| ASO | | Exon skipping | | | iency | ASO | | Exon skipping | | | iency | ASO | | Exon skipping | | | iency |
| treat- | | efficiency | | | (com- | treat- | | efficiency | | | (com- | treat- | | efficiency | | | (com- |
| ment | | | | Partial | pared | ment | | | | Partial | pared | ment | | | | Partial | pared |
| concen- | | Full | Exon-2 | exon-2 | to un- | concen- | | Full | Exon-2 | exon-2 | to un- | concen- | | Full | Exon-2 | exon-2 | to un- |
| tration | | length | skipped | skipped | treated) | tration | | length | skipped | skipped | treated) | tration | | length | skipped | skipped | treated) |
| 400 | nM | 86% | 14% | 0% | 79% | 400 | nM | 31% | 69% | 0% | 90% | 400 | nM | 100% | 0% | 0% | 92% |
| 200 | nM | 87% | 13% | 0% | 66% | 200 | nM | 21% | 79% | 0% | 93% | 200 | nM | 100% | 0% | 0% | 86% |
| 100 | nM | 91% | 9% | 0% | 63% | 100 | nM | 47% | 53% | 0% | 82% | 100 | nM | 100% | 0% | 0% | 58% |
| 50 | nM | 97% | 3% | 0% | 60% | 50 | nM | 68% | 32% | 0% | 84% | 50 | nM | 100% | 0% | 0% | 42% |
| 25 | nM | 100% | 0% | 0% | 57% | 25 | nM | 92% | 8% | 0% | 72% | 25 | nM | 100% | 0% | 0% | 42% |
| 12.5 | nM | 100% | 0% | 0% | 48% | 12.5 | nM | 100% | 0% | 0% | 45% | 12.5 | nM | 100% | 0% | 0% | 36% |

TABLE 3C
LIN28B RNA inhibition efficacy of different chemistries of ASO-5, evaluated by dose dependency test in liver cancer HepG2 cells
C. ASO-5 LIN28B 1E2A (+69 +93)

| C1. 2′-OMe-PS | | | | | | C2. 2′-MOE-PS | | | | | | C3. 7-11-7 MOE gapmer | | | | | |
|---|---|---|---|---|---|---|---|---|---|---|---|---|---|---|---|---|---|
| | | | | | Full | | | | | | Full | | | | | | Full |
| | | | | | length | | | | | | length | | | | | | length |
| | | | | | knock- | | | | | | knock- | | | | | | knock- |
| | | | | | down | | | | | | down | | | | | | down |
| | | | | | effic- | | | | | | effic- | | | | | | effic- |
| ASO | | Exon skipping | | | iency | ASO | | Exon skipping | | | iency | ASO | | Exon skipping | | | iency |
| treat- | | efficiency | | | (com- | treat- | | efficiency | | | (com- | treat- | | efficiency | | | (com- |
| ment | | | | Partial | pared | ment | | | | Partial | pared | ment | | | | Partial | pared |
| concen- | | Full | Exon-2 | exon-2 | to un- | concen- | | Full | Exon-2 | exon-2 | to un- | concen- | | Full | Exon-2 | exon-2 | to un- |
| tration | | length | skipped | skipped | treated) | tration | | length | skipped | skipped | treated) | tration | | length | skipped | skipped | treated) |
| 400 | nM | 100% | 0% | 0% | 30% | 400 | nM | 100% | 0% | 0% | 65% | 400 | nM | 100% | 0% | 0% | 48% |
| 200 | nM | 100% | 0% | 0% | 9% | 200 | nM | 100% | 0% | 0% | 42% | 200 | nM | 100% | 0% | 0% | 38% |
| 100 | nM | 100% | 0% | 0% | 5% | 100 | nM | 100% | 0% | 0% | 34% | 100 | nM | 100% | 0% | 0% | 17% |
| 50 | nM | 100% | 0% | 0% | 0% | 50 | nM | 100% | 0% | 0% | 19% | 50 | nM | 100% | 0% | 0% | 12% |
| 25 | nM | 100% | 0% | 0% | 0% | 25 | nM | 100% | 0% | 0% | 22% | 25 | nM | 100% | 0% | 0% | 0% |
| 12.5 | nM | 100% | 0% | 0% | 0% | 12.5 | nM | 100% | 0% | 0% | 22% | 12.5 | nM | 100% | 0% | 0% | 0% |

TABLE 3D
LIN28B RNA inhibition efficacy of different chemistries of ASO-6, evaluated by dose dependency test in liver cancer HepG2 cells
D. ASO-5 LIN28B 1E2A (+142 +166)

| D1. 2′-OMe-PS | | | | | | D2. 2′-MOE-PS | | | | | | D3. 7-11-7 MOE gapmer | | | | | |
|---|---|---|---|---|---|---|---|---|---|---|---|---|---|---|---|---|---|
| | | | | | Full | | | | | | Full | | | | | | Full |
| | | | | | length | | | | | | length | | | | | | length |
| | | | | | knock- | | | | | | knock- | | | | | | knock- |
| | | | | | down | | | | | | down | | | | | | down |
| | | | | | effic- | | | | | | effic- | | | | | | effic- |
| ASO | | Exon skipping | | | iency | ASO | | Exon skipping | | | iency | ASO | | Exon skipping | | | iency |
| treat- | | efficiency | | | (com- | treat- | | efficiency | | | (com- | treat- | | efficiency | | | (com- |
| ment | | | | Partial | pared | ment | | | | Partial | pared | ment | | | | Partial | pared |
| concen- | | Full | Exon-2 | exon-2 | to un- | concen- | | Full | Exon-2 | exon-2 | to un- | concen- | | Full | Exon-2 | exon-2 | to un- |
| tration | | length | skipped | skipped | treated) | tration | | length | skipped | skipped | treated) | tration | | length | skipped | skipped | treated) |
| 400 | nM | 24% | 4% | 72% | 80% | 400 | nM | 15% | 44% | 41% | 91% | 400 | nM | 86% | 0% | 14% | 62% |
| 200 | nM | 25% | 6% | 69% | 76% | 200 | nM | 16% | 41% | 43% | 88% | 200 | nM | 82% | 0% | 18% | 58% |
| 100 | nM | 44% | 6% | 50% | 59% | 100 | nM | 33% | 23% | 44% | 59% | 100 | nM | 82% | 0% | 18% | 44% |
| 50 | nM | 63% | 1% | 36% | 50% | 50 | nM | 49% | 14% | 37% | 37% | 50 | nM | 89% | 0% | 11% | 40% |
| 25 | nM | 89% | 0% | 11% | 11% | 25 | nM | 70% | 6% | 25% | 35% | 25 | nM | 92% | 0% | 8% | 26% |
| 12.5 | nM | 98% | 0% | 2% | 27% | 12.5 | nM | 80% | 3% | 17% | 16% | 12.5 | nM | 97% | 0% | 3% | 26% |

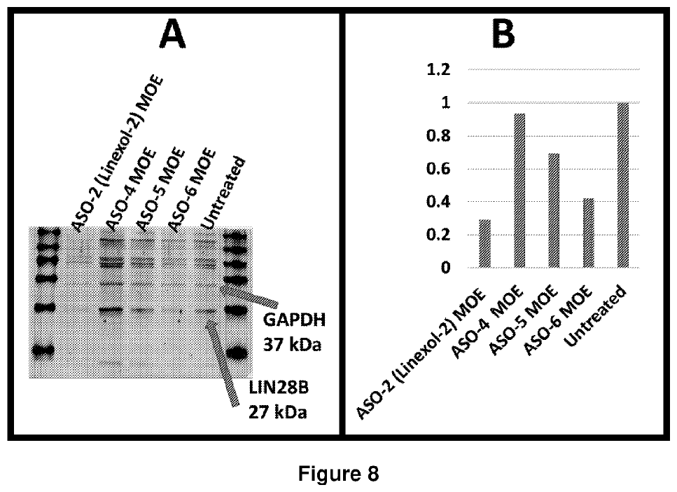

The efficacy of LIN28B protein inhibition by 2′-MOE-PS form of ASO-2, ASO-4, ASO-5, and ASO-6 were evaluated by Western blot analysis (FIG. 8). Based on the results, ASO-2 induced as much as 71% of LIN28B protein reduction, followed by ASO-6 (58%), ASO-5 (31%), and ASO-4 (7%), compared to the HepG2 cells without ASO treatment (FIG. 8).

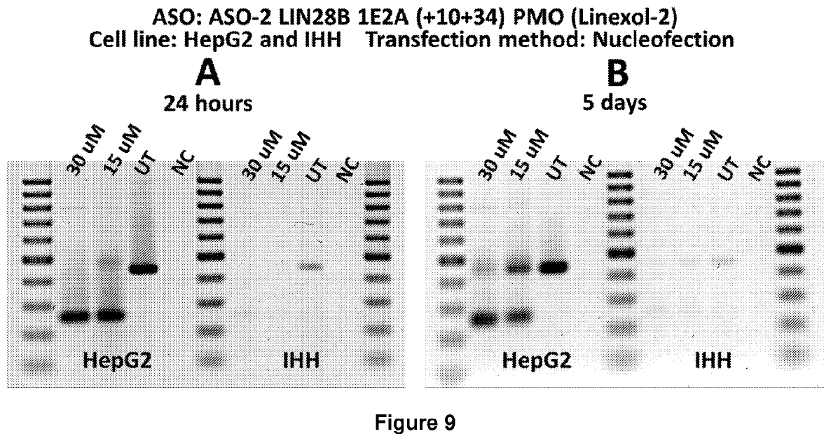

Further, the efficacy of LIN28B mRNA inhibition by PMO form of ASO-2 was evaluated via RT-PCR and agarose gel analysis (FIG. 9). Based on the results, different concentrations (30 μM, 15 μM) of PMO form of ASO-2 induced 100% exon-2 skipping after 24 hours of nucleofection (FIG. 9A) and 79% (30 μM), 53% (15 μM) of exon-2 skipping after 5 days of nucleofection (FIG. 9B).

### Example 4

### Evaluation of Cell Viability Using WST-Dye-Based Assay

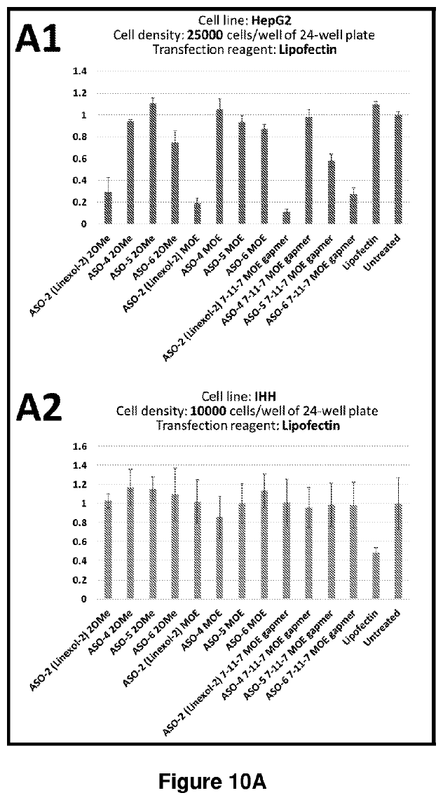

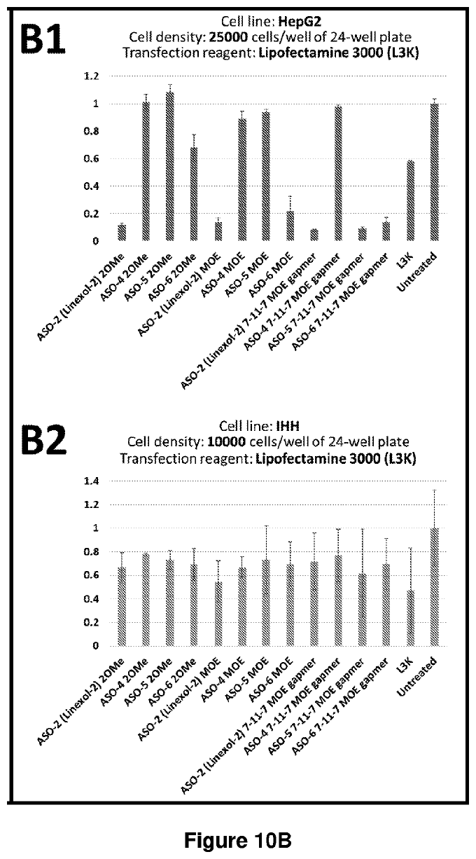

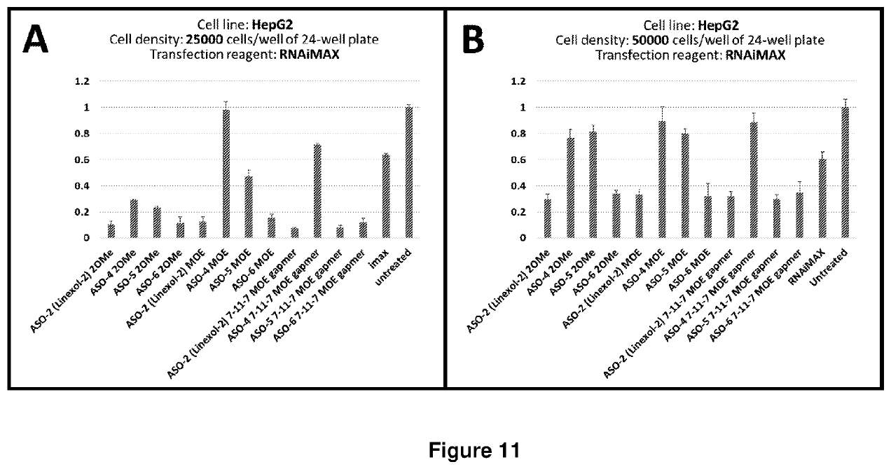

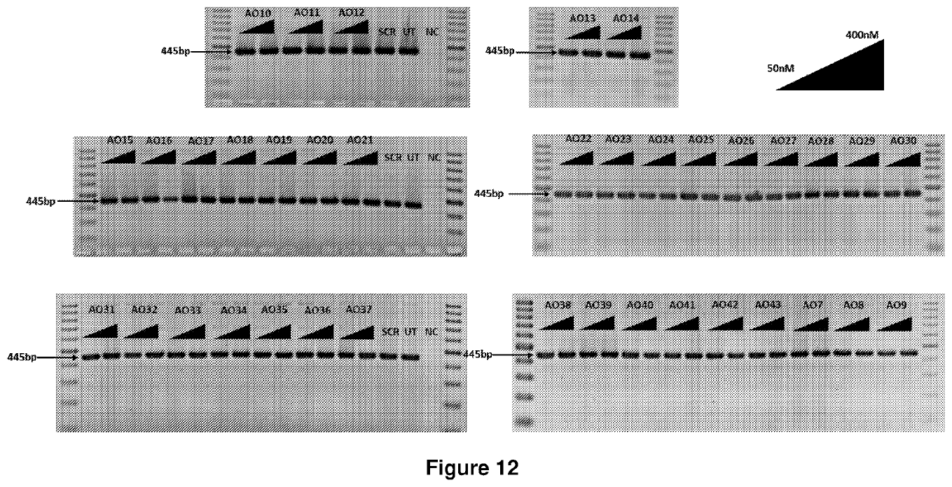

WST-dye based cell viability assay is an established method for analysing the efficacy of drug molecule's potential to inhibit the proliferation of cancer cells in vitro. In line with this, we have tested ASO-2, ASO-4, ASO-5, and ASO-6 candidates for their potential to inhibit the proliferation of cancer cells in vitro. Remarkably, our results showed that ASO-2 and ASO-6 were able to inhibit the proliferation of liver cancer cells efficiently compared to the transfection reagent control and untreated samples (FIG. 10 A1, B1; FIG. 11). In contrast, the ASOs did not induce significant inhibition of proliferation of normal human liver cells (IHH cells) (FIG. 10 A2, B2).

1. An isolated or purified antisense oligomer for modifying pre-mRNA splicing in the LIN28B gene transcript or part thereof which has a modified backbone structure and sequences with at least 95% sequence identity to the isolated or purified antisense oligomer which have a modified backbone structure for modifying pre-mRNA splicing, and/or induction of RNase H, and/or translational blockage in the LIN28B gene transcript or part thereof.

2. The antisense oligomer of claim 1 that induces non-productive splicing or functional impairment, and/or degradation of mRNA, and/or inhibition of translational process in the LIN28B gene transcript or part thereof.

3. The antisense oligomer of claim 1 selected from the list comprising: SEQ ID NOs: 1-43.

4. The antisense oligomer of claim 1 wherein the antisense oligomer contains one or more nucleotide positions subject to an alternative chemistry or modification chosen from the list comprising: (i) modified sugar moieties; (ii) resistance to RNase H; (iii) oligomeric mimetic chemistry.

5. The antisense oligomer of claim 1 wherein the antisense oligomer is further modified by: (i) chemical conjugation to a moiety; and/or (ii) tagging with a cell penetrating peptide.

6. The antisense oligomer of claim 1 wherein the antisense oligomer is a 2′-O-methyl phosphorothioate (2′-OMe-PS) oligomer, 2′-O-methoxyethyl phosphorothioate (2′-MOE-PS) oligomer or 7-11-7 MOE gapmer.

7. The antisense oligomer of claim 1 wherein the antisense oligomer is a phosphorodiamidate morpholino oligomer (PMO).

8. The antisense oligomer of claim 1 wherein when any uracil (U) is present in the nucleotide sequence, the uracil (U) is replaced by a thymine (T).

9. The antisense oligomer of claim 1 that operates to induce skipping of one or more of the exons or parts of the exons of the LIN28B gene transcript or part thereof.

10. The antisense oligomer of claim 1 that operates to induce translational blockage of the LIN28B gene transcript or part thereof.

11. The antisense oligomer of claim 1 that operates to induce RNase H mediated degradation of the LIN28B gene transcript or part thereof.

12. A method for manipulating splicing in a LIN28B gene transcript, and/or induction of RNase H, and/or translational blockage, the method including the step of:
a) providing one or more of the antisense oligomers according to any one of claims 1 to 11 and allowing the oligomer(s) to bind to a target nucleic acid site.

13. A pharmaceutical, prophylactic, or therapeutic composition to treat or ameliorate the effects of a cancer associated with LIN28B expression in a subject, the composition comprising:
a) one or more antisense oligomers according to any one of claims 1 to 11, and
b) one or more pharmaceutically acceptable carriers and/or diluents.

14. A method to treat or ameliorate the effects of a cancer associated with LIN28B expression, comprising the step of:
a) administering to the subject an effective amount of one or more antisense oligomers or pharmaceutical composition comprising one or more antisense oligomers according to any one of claims 1 to 11.

15. The use of purified and isolated antisense oligomers according to any one of claims 1 to 11, for the manufacture of a medicament to treat or ameliorate the effects of a cancer associated with LIN28B expression.

16. A kit to treat or ameliorate the effects of a cancer associated with LIN28B expression in a subject, which kit comprises at least an antisense oligomer according to any one of claims 1 to 11 and combinations or cocktails thereof, packaged in a suitable container, together with instructions for its use.

17. The method of claim 12 or 14, composition of claim 13 use of claim 15 or kit of claims 16 wherein the LIN28B expression related cancer is a solid tumour cancer related to LIN28B expression, preferably a solid tumour cancer chosen from the list comprising: liver cancer, lung cancer, head and neck cancer, stomach cancer, adenocarcinoma, epithelial ovarian cancer, colorectal cancer, urothelial cancer, breast cancer, cervical cancer, endometrial cancer, brain cancer, colon cancer, acute myeloid leukemia, atypical teratoid tumour, oesophageal cancer, medulloblastoma, multiple myeloma, neuroblastoma, oral squamous cell carcinoma, Wilms tumour, and prostate cancer.

18. The method of claim 12 or 14, composition of claim 13 use of claim 15 or kit of claims 16 wherein the subject with the cancer associated with LIN28B expression is a human.

19. The antisense oligomer of claim 1 selected from the list comprising: SEQ ID NOs: 2, 4, 5 and 6.

20. The antisense oligomer of claim 1 that is SEQ ID NO: 2.


SEQUENCE LISTING (47 sequences)

SEQ ID NO: 1 - DNA, 25 bp, Homo sapiens
ctttgctagccccgcctgagaagga

SEQ ID NO: 2 - DNA, 25 bp, Homo sapiens
ccgggctcttctccaccacctttgc

SEQ ID NO: 3 - DNA, 25 bp, Homo sapiens
tcctcctctgccggctccggcagct

SEQ ID NO: 4 - DNA, 25 bp, Homo sapiens
cctgggattcctcctctgccggctc

SEQ ID NO: 5 - DNA, 25 bp, Homo sapiens
tacagtggccagttccgcgcaaaac

SEQ ID NO: 6 - DNA, 25 bp, Homo sapiens
ggaatatccaaggggcttccctctc

SEQ ID NO: 7 - DNA, 25 bp, Homo sapiens
catgaatagtttgctctgtaaagag

SEQ ID NO: 8 - DNA, 25 bp, Homo sapiens
ttcttttaggcttctaaatccttcc

SEQ ID NO: 9 - DNA, 25 bp, Homo sapiens
taaatgtgaattccactggttctcc

SEQ ID NO: 10 - DNA, 25 bp, Homo sapiens
gactcaaggcctttggaagattttt

SEQ ID NO: 11 - DNA, 25 bp, Homo sapiens
cccaccaggtcctgttacccgtatt

SEQ ID NO: 12 - DNA, 25 bp, Homo sapiens
ttctttcacttcctaaacaggggct

SEQ ID NO: 13 - DNA, 25 bp, Homo sapiens
ttctgtagtgtcttccctttgggtc

SEQ ID NO: 14 - DNA, 25 bp, Homo sapiens
ttacctatctccctttggttttctt

SEQ ID NO: 15 - DNA, 25 bp, Homo sapiens
ggtctaatgtgctttccttcttttg

SEQ ID NO: 16 - DNA, 25 bp, Homo sapiens
caaatttagctcgcatggtctaatg

SEQ ID NO: 17 - DNA, 25 bp, Homo sapiens
acatcttgattttgtgcgatcataa

SEQ ID NO: 18 - DNA, 25 bp, Homo sapiens
cggagtgatcttctgcatcaatcta

SEQ ID NO: 19 - DNA, 25 bp, Homo sapiens
tgagatgaaaactttccctttggaa

SEQ ID NO: 20 - DNA, 25 bp, Homo sapiens
ccacgggccctcagctccaaactcg

SEQ ID NO: 21 - DNA, 25 bp, Homo sapiens
cttcggccatgttgccccacgggcc

SEQ ID NO: 22 - DNA, 25 bp, Homo sapiens
tagcatgatgatcaaggccaccaca

SEQ ID NO: 23 - DNA, 25 bp, Homo sapiens
aggtagactacattccttagcatga

SEQ ID NO: 24 - DNA, 25 bp, Homo sapiens
atgtacagccatgcccgcctcccac

SEQ ID NO: 25 - DNA, 25 bp, Homo sapiens
ctagcctcctgaggaaacggtggtg

SEQ ID NO: 26 - DNA, 25 bp, Homo sapiens
tcacacatgcacatattgacagaca

SEQ ID NO: 27 - DNA, 25 bp, Homo sapiens
cacacagactcaggctctctccctc

SEQ ID NO: 28 - DNA, 25 bp, Homo sapiens
atataaaaatcctcatgtacacaca

SEQ ID NO: 29 - DNA, 25 bp, Homo sapiens
attactactccagagatttcaagaa

SEQ ID NO: 30 - DNA, 25 bp, Homo sapiens
ctgccttcaaaaaagggggaaaaaa

SEQ ID NO: 31 - DNA, 25 bp, Homo sapiens
agttaaggtactgccttcaaaaaag

SEQ ID NO: 32 - DNA, 25 bp, Homo sapiens
tcaaattgcacccataaagaccata

SEQ ID NO: 33 - DNA, 25 bp, Homo sapiens
gtctggtaagatgatgaaaaggatt

SEQ ID NO: 34 - DNA, 25 bp, Homo sapiens
ctcttagtttagtctggtaagatga

SEQ ID NO: 35 - DNA, 25 bp, Homo sapiens
ttatgtaatagtgataatattctca

SEQ ID NO: 36 - DNA, 25 bp, Homo sapiens
agcaaagttctttgtcctgagtatg

SEQ ID NO: 37 - DNA, 25 bp, Homo sapiens
cattaagacatctagaaaattaaag

SEQ ID NO: 38 - DNA, 25 bp, Homo sapiens
gttcttctctaaaacaaataaatct

SEQ ID NO: 39 - DNA, 25 bp, Homo sapiens
gtttgatgcttttaacaagatgttc

SEQ ID NO: 40 - DNA, 25 bp, Homo sapiens
catcattttcaagctggtaactctc

SEQ ID NO: 41 - DNA, 25 bp, Homo sapiens
atgtgattcaagaggtagtcaacac

SEQ ID NO: 42 - DNA, 25 bp, Homo sapiens
ggtggtaggtgccagtggttgatag

SEQ ID NO: 43 - DNA, 25 bp, Homo sapiens
taattgaagccagcttggtggtagg

SEQ ID NO: 44 - DNA, 20 bp, Homo sapiens
tcacgagtttggagctgagg

SEQ ID NO: 45 - DNA, 20 bp, Homo sapiens
ttcctaaacaggggctccca

SEQ ID NO: 46 - DNA, 20 bp, Homo sapiens
gcacattagaccatgcgagc

SEQ ID NO: 47 - DNA, 20 bp, Homo sapiens
ctcccaccaggtcctgttac